(19) United States
(12) Patent Application Publication
(10) Pub. No.: US 2009/0246163 A1
(43) Pub. Date: Oct. 1, 2009

(54) ANTI-STATIC SKIN PRODUCTS AND METHOD

(75) Inventor: Ashok Wahi, Hillsborough, NJ (US)

Correspondence Address: STANLEY H. KREMEN, 4 LENAPE LANE, EAST BRUNSWICK, NJ, 08816, US

(73) Assignee: TRUTEK CORP., Hillsborough, NJ (US)

(21) Appl. No.: 12/475,719

(22) Filed: Jun. 1, 2009

Related U.S. Application Data
(60) Provisional application No. 61/091,931, filed on Aug. 26, 2008.
(60) Provisional application No. 61/102,011, filed on Oct. 2, 2008.

(51) Int. Cl. A61K 31/785

(52) U.S. Cl. 424/78.03

(57) ABSTRACT

An aqueous or non-aqueous preparation comprising cationic agents to neutralize a negative charge thereby relieving tension in the skin and preventing moisture from escaping. It relieves static-tension in the skin thereby reducing the distance between the pores. The difference between the Present Invention and other water based skin conditioners is the amount of cationic agents used. The Present Invention utilizes much more of these ingredients than other commercial products. This produces the unexpected result of neutralizing the charge on the skin and relieving tension, softening and moisturizing the skin. In its most basic form, it contains no moisturizing or other skin conditioning agents. These products can be added to the Present Invention, or alternatively, the Present Invention may be used as an additive to most currently available commercial aqueous and non-aqueous based skin moisturizers for a synergistic skin softening effect.

### CROSS REFERENCE TO RELATED APPLICATIONS

a) The Present Application is the non-provisional counterpart of U.S. Provisional Patent Application Ser. No. 61/102,011 filed on Oct. 2, 2008, which is incorporated by reference in its entirety herein.
b) The Present Application is related to my pending U.S. patent application Ser. No. 12/467,271 filed on May 16, 2009, which is incorporated by reference in its entirety herein.
c) The Present Application is related to my pending U.S. patent application Ser. No. 12/466,382 filed on May 14, 2009, which is incorporated by reference in its entirety herein.
d) The Present Application is related to my pending U.S. Provisional patent application Ser. No. 10/161,821 filed on Jun. 4, 2002, which is incorporated by reference in its entirety herein.
e) The Present Application is related to my U.S. Pat. No. 5,468,488, entitled “ELECTROSTATICALLY CHARGED NASAL APPLICATION PRODUCT AND METHOD” issued on Nov. 21, 1995. This patent is incorporated by reference in its entirety herein.
f) The Present Application is related to my U.S. Pat. No. 5,674,481, entitled “ELECTROSTATICALLY CHARGED NASAL TOPICAL APPLICATION PRODUCT” issued on Oct. 7, 1997. This patent is incorporated by reference in its entirety herein.
g) The Present Application is related to my U.S. Pat. No. 6,844,005 entitled “ELECTROSTATICALLY CHARGED NASAL APPLICATION PRODUCT WITH INCREASED STRENGTH” issued on Jan. 18, 2005. This patent is incorporated by reference in its entirety herein.

### FIELD OF THE INVENTION

The Present Invention relates to the field of protective compositions to condition dry, cracked, and chapped skin so as to retain moisture, and to condition hair.

### BACKGROUND OF THE INVENTION

There is currently a wide variety of moisturizing skin softeners on the market. The primary purpose of such products is to prevent moisture from escaping from the skin while adding moisture to the skin. The vast majority of these commercial products are oil based, or they utilize an oil-water emulsion. The effect of these oil based products is to coat the skin with a layer that is water impermeable. This water impermeable layer prevents the skin from losing water due to evaporation. Most of these preparations use various additives that treat skin to make it smooth, moist, and supple. Yet, the skin cannot breathe through this impermeable layer. This represents a problem in that oxygen must be absorbed through the skin to sustain life.

Better moisturizing skin conditioners are water based, but these are in the minority. They employ moisturizing and conditioning agents to treat dry and damaged skin. Some of them utilize a very small amount of cationic agent in order to minimize the electrostatic effect of using these products. Cationic additives are normally not used in oil based preparations. Unlike oil based preparations that work by placing a protective layer over the skin, water based preparations work by reducing skin tension.

If the air is very dry, a person moving through air will accumulate negatively charged electrons on his skin due to air/skin friction. Should enough electrons accumulate on the skin, a person's hair will “stand on end.” Having a “like” electrical static charge, the hairs repel each other. Skin cells exhibit a similar phenomenon. For example, “cracking” of the skin results from dry skin cells “standing on end,” and repelling one another. Dry, chapped, or broken skin is due to a repulsive force between the cells. This produces skin tension. The condition is aggravated by a lack of moisture. The pores open, and moisture escapes. Rubbing one's hands together also creates an electrostatic charge that makes the skin dry and chapped. The skin has accumulated an excess of electrons.

Hair conditioners work in a different way. When a person wets his or her hair, the hair tends to “go limp” because water is a plasticizing agent for hair. Hair conditioners enhance the plasticizing effect. In addition, they add ingredients to treat hair to make it less brittle.

### SUMMARY OF THE INVENTION

The Present Invention is an aqueous preparation that comprises cationic agents to neutralize a negative charge thereby relieving tension in the skin, allowing the pores to close, and preventing moisture from escaping. It reduces the distance between the pores. The difference between the Present Invention and other water based skin conditioners is the amount of cationic agents used. The Present Invention utilizes much more of these ingredients than other commercial products. This produces the unexpected result of neutralizing the charge on the skin and relieving tension. In its most basic form, it contains no moisturizing or other skin conditioning agents. These products can be added to the Present Invention, or alternatively, the Present Invention may be used as an additive to commercial water based skin softeners.

### DETAILED DESCRIPTION OF THE INVENTION

A typical formulation of the product of the Present Invention might consist of:

| | |
|---|---|
| Celquat SC-240c (Polyquaternium-10) | 0-4% |
| Merquat 100 | 0-11% |

as the principal ingredients (cationic agents) and:
Distilled Water
Propylene Glycol
Tetrasodium EDTA
Polywax
Glycerol Monostearate (Glyceryl Stearate)
Vanilla
Glycerin
Germall Plus (Diazolidinyl Urea & Isopropynyl Butylcarbamate)

This formulation is typical. Merquat 280 is Polyquaternium-22. Celquat and Merquat are both strong cationic agents. The formulations created thus far comprised both Celquat and Merquat. However, it could be useful to have either agent be the sole active ingredient.

Cationic agents other than Celquat and Merquat are known in the art. These other agents are also useful in the Present Invention either as a substitute for Celquat and Merquat or as an additive. As mentioned before, only water based products can utilize cationic agents. The difference between the Present Invention and other commercial conditioners is the strength of the cationic effect. This produces the unexpected result of neutralizing the negative charge of the skin, thereby reducing skin tension (by allowing the skin cells to reside closer to each other), and closing pores so as to retain moisture.

The basic formulation mainly comprises the cationic agents in an aqueous base. However, moisturizing and conditioning agents can be added to the basic formulation. On the other hand, the basic formulation may be added to other water based skin conditioners to enhance their effect.

The Present Invention may be used as a treatment for psoriasis which is aggravated by dry skin. It may also be used to treat chafed elbows. In addition, when added to hair conditioners, it enhances the plasticizing effect by neutralizing any negative charge retained by the hair.

Other formulations comprise the following ingredients:

Water

Cetyl Alcohol

Cetrimonium Chloride

Polyquaternium-67
Glyceryl Stearate

PEG-100 Stearate

Polyquaternium-22

Phenoxyethanol

Stearyl Alcohol

Benzalkonium Chloride

Hydroxypropyl Trimonium Hydrolyzed Soy

Methylparaben

Propylparaben

Butylparaben

Ethylparaben

Isobutylparaben

Sodium Hydroxide

Hydrolyzed Algin

Chlorella Vulgaris Extract

Sea Water

Propylene Glycol

Euterpe Oleracea (Acai) Fruit Extract

Lycium Barbarum (Goji Berry) Fruit Extract

Punica Granatum (Pomegranate) Extract

Diospyros Kaki (Persimmon) Fruit Extract

Fragrance

Yet, other formulations comprise:

Water

Isocetyl Behenate

Cetyl Alcohol

Cetrimonium Chloride

Polyquaternium-67

Glyceryl Stearate

PEG-100 Stearate

Polyquaternium-22

Phenoxyethanol

Stearyl Alcohol

Benzalkonium Chloride

Hydroxypropyl Trimonium Hydrolyzed Soy

Methylparaben

Propylparaben

Butylparaben

Ethylparaben

Isobutylparaben

Sodium Hydroxide

Propylene Glycol

Symphytum Officinale (Comfrey) Leaf Extract

Centaurea Cyanus (Cornflower) Flower Extract

Echinacea Angustifolia Leaf Extract

Silybum Marianum (Milk Thistle) Extract

Fragrance

The formulations that follow comprise these generic ingredients:

water (used as a solvent and moisturizer);

at least one quaternary conditioner;

at least one preservative;

at least one thickener;

an emollient;

an emulsifier; and

a neutralizing agent to adjust pH as desired.

The above formulation has an aqueous base, but one skilled in the art would know that the formulation may also be non-aqueous. In addition, some embodiments comprise an antiseptic, an anti-inflammatory agent, an antioxidant, an astringent, and an agent to promote skin healing, having cleansing, antibacterial and antiseptic attributes. Also, a fragrance would probably be added.

The quaternary conditioners that are used include (at least one must be used), without limitation:

Polyquaternium-22;

Polyquaternium-67;

Cetyl Trimethyl Ammonium Chloride;

Hydroxypropyl Trimonium Hydrolyzed Soy Protein; and

Benzalkonium Chloride.

The preservatives used include, without limitation:

Phenoxyethanol.

Methyloaraben,

Butylparben,

Ethylparaben,

Propylparaben,

Isobutylpareben, and

Phenoxyethanol.

The thickeners used include, without limitation, Cetyl Alcohol, and Stearyl Alcohol. The emollients used include, without limitation:

Isocetyl Behanate,

Water,

Hydrolyzed Algin,

Chlorella Vulgaris, and

C10-30 Cholesterol/Lanosterol Esters.

The emulsifiers used include, without limitation, Glyceryl Stearate, and PEG-100 Stearate. The neutralizing agent used was a 5% solution of sodium hydroxide. Fragrances used were lavender, sandalwood, and honeysuckle.

Tables 1 through 10 (infra) provide examples of these formulations:

TABLE 1

| Ingredient | Percent | Functionality |
|---|---|---|
| Water | 82%-94% | Solvent, Mois- |
| | | turizer |
| Polyquaternium - 67 | 0.5%-2% | Conditioner/ |
| | | Quat |
| Phenoxyethanol, Methyloaraben, | 1% | Preservative |
| Butylparben, Ethylparaben, | | |
| Propylparaben, Isobutylpareben | | |
| Polyquaternium 22 | 0.25%-1% | Conditioner/ |
| | | Quat |
| Cetyl Trimethyl Ammonium Chloride | 1%-3% | Conditioner/ |
| | | Quat |
| Cetyl Alcohol | 1%-3% | Thickener |
| C10-30 Cholesterol/Lanosterol | 0.5%-2% | Emollient |
| Esters | | |
| Glyceryl Stearate, PEG-100 Stearate | 1%-3% | Emulsifier |
| Hydroxypropyl Trimonium Hydrolyzed | 0.25% 1% | Conditioner/ |
| Soy | | Quat |
| Protein | | |
| Benzylkonium Chloride | 0.5%-1.5% | Conditioner/ |
| | | Quat/Neut. |
| | | Agent |
| Sodium Hydroxide | 0.01%-0.02% | Neutralizing |
| | | agent |

TABLE 2

| Ingredient | Percent | Functionality |
|---|---|---|
| Water | 81%-92% | Solvent, Mois- |
| | | turizer |
| Isoprene Glycol | 1% | |
| Polyquaternium - 67 | 0.5%-2% | Conditioner/ |
| | | Quat |
| Phenoxyethanol, Methyloaraben, | 1% | Preservative |
| Butylparben, Ethylparaben, | | |
| Propylparaben, Isobutylpareben | | |
| Polyquaternium 22 | 0.2%-1% | Conditioner/ |
| | | Quat |
| Cetyl Trimethyl Ammonium Chloride | 1%-3% | Conditioner/ |
| | | Quat |
| Cetyl Alcohol | 1%-3% | Thickener |
| C10-30 Cholesterol/Lanosterol | 0.6% | Emollient |
| Esters | | |
| Macadamia Nut Oil | 0.4% | |
| Glyceryl Stearate, PEG-100 Stearate | 1.5%-3% | Emulsifier |
| Hydroxypropyl Trimonium Hydrolyzed | 0.25%-0.75% | Conditioner/ |
| Soy Protein | | Quat |
| Pheohydrane | 1% | |
| Benzylkonium Chloride | 0.5%-1.5% | Conditioner/ |
| | | Quat/Neut. |
| | | Agent |
| Sodium Hydroxide | 0.02% | Neutralizing |
| | | agent |

TABLE 3

| Ingredient | Percent | Functionality |
|---|---|---|
| Water | 80%-93% | Solvent, Mois- |
| | | turizer |
| Polyquaternium - 67 | 1%-3% | Conditioner/ |
| | | Quat |
| Phenoxyethanol, Methyloaraben, | 1% | Preservative |
| Butylparben, Ethylparaben, | | |
| Propylparaben, Isobutylpareben | | |
| Polyquaternium 22 | 0.1%-0.5% | Conditioner/ |
| | | Quat |
| Cetyl Trimethyl Ammonium Chloride | 1%-3% | Conditioner/ |
| | | Quat |
| Stearyl Alcohol | 0.1%-1% | Thickener |
| Cetyl Alcohol | 2%-4% | Thickener |
| Water, Hydrolyzed Algin, Chlorella | 0.6%-1.4% | Emollient, mois- |
| Vulgaris | | turizer |
| Extract, Sea Water | Trace | |
| Glyceryl Stearate, PEG-100 Stearate | 1%-3% | Emulsifier |
| Hydroxypropyl Trimonium Hydrolyzed | 0.1%-1% | Conditioner/ |
| Soy | | Quat |
| Benzylkonium Chloride | 0.5%-1.5% | Conditioner/ |
| | | Quat/Neut. |
| | | Agent |
| Sodium Hydroxide | 0.01% | Neutralizing |
| | | agent |

TABLE 4

| Ingredient | Percent | Functionality |
|---|---|---|
| Water | 82%-90% | Solvent, Mois- |
| | | turizer |
| Polyquaternium - 67 | 1%-2% | Conditioner/ |
| | | Quat |
| Phenoxyethanol, Methyloaraben, | 1% | Preservative |
| Butylparben, Ethylparaben, | | |
| Propylparaben, Isobutylpareben | | |
| Polyquaternium 22 | 0.1%-1% | Conditioner/ |
| | | Quat |
| Cetyl Trimethyl Ammonium Chloride | 1%-4% | Conditioner/ |
| | | Quat |
| Stearyl Alcohol | 0.5 | Thickener |
| Cetyl Alcohol | 3% | Thickener |
| Water, Hydrolyzed Algin, Chlorella | 1% | Emollient, mois- |
| Vulgaris | | turizer |
| Extract, Sea Water | Trace | |
| Glyceryl Stearate, PEG-100 Stearate | 2%-3% | Emulsifier |
| Phenoxyethanol | Trace | Preservative |
| Hydroxypropyl Trimonium Hydrolyzed | 0.1%-1% | Conditioner/ |
| Soy | | Quat |
| Propylene Glycol, Euterpe Oleracea, | 1% | Antiseptic, Anti- |
| (Acai) Fruit Extract, Lycium Barbarum | | inflammatory, |
| (Goji Berry) Fruit Extract, Punica | | Antioxidant, |
| Granatum (Pomegranate) Extract, | | Astringent |
| Diospyros, Kaki (Persimmon) Fruit | | |
| Extract | | |
| Benzylkonium Chloride | 0.5%-1.2% | Conditioner/ |
| | | Quat/Neut. |
| | | Agent |
| Sodium Hydroxide | 0.02 | Neutralizing |
| | | agent |
| Fragrance | 0.5 | Fragrance |

TABLE 5

| Ingredient | Percent | Functionality |
|---|---|---|
| Water | 82%-88% | Solvent, Mois- |
| | | turizer |
| Polyquaternium - 67 | 1%-2% | Conditioner/ |
| | | Quat |
| Phenoxyethanol, Methyloaraben, | 1% | Preservative |
| Butylparben, Ethylparaben, | | |
| Propylparaben, Isobutylpareben | | |
| Polyquaternium 22 | 0.1%-1% | Conditioner/ |
| | | Quat |
| Cetyl Trimethyl Ammonium Chloride | 1%-3% | Conditioner/ |
| | | Quat |
| Stearyl Alcohol | 0.5% | Thickener |
| Cetyl Alcohol | 3% | Thickener |
| Water, Hydrolyzed Algin, Chlorella | 1% | Emollient, mois- |
| Vulgaris | | turizer |
| Extract, Sea Water | Trace | |
| Glyceryl Stearate, PEG-100 Stearate | 2.5% | Emulsifier |
| Phenoxyethanol | Trace | Preservative |
| Hydroxypropyl Trimonium Hydrolyzed | 0.2%-0.75% | Conditioner/ |
| Soy | | Quat |
| Propylene Glycol, Euterpe Oleracea, | 1% | Antiseptic, Anti- |
| (Acai) Fruit Extract, Lycium Barbarum | | inflammatory, |
| (Goji Berry) Fruit Extract, Punica | | Antioxidant, |
| Granatum (Pomegranate) Extract, | | Astringent |
| Diospyros, Kaki (Persimmon) Fruit | | |
| Extract | | |
| Benzylkonium Chloride | 0.5%-1.5% | Conditioner/ |
| | | Quat/Neut. |
| | | Agent |
| Sodium Hydroxide | 0.01% | Neutralizing |
| | | agent |
| Fragrance | 0.5% | Fragrance |

TABLE 6

| Ingredient | Percent | Functionality |
|---|---|---|
| Water | 76%-93% | Solvent, Mois- |
| | | turizer |
| Polyquaternium - 67 | 1%-2% | Conditioner/ |
| | | Quat |
| Phenoxyethanol, Methyloaraben, | 1% | Preservative |
| Butylparben, Ethylparaben, | | |
| Propylparaben, Isobutylpareben | | |
| Polyquaternium 22 | 0.1%-1.2% | Conditioner/ |
| | | Quat |
| Cetyl Trimethyl Ammonium Chloride | 1%-3% | Conditioner/ |
| | | Quat |
| Stearyl Alcohol | 0.1%-1% | Thickener |
| Cetyl Alcohol | 1%-4% | Thickener |
| Water, Hydrolyzed Algin, Chlorella | 0.5%-1.5% | Emollient, mois- |
| Vulgaris | | turizer |
| Extract, Sea Water | Trace | |
| Glyceryl Stearate, PEG-100 Stearate | 1.5%-4% | Emulsifier |
| Phenoxyethanol | Trace | Preservative |
| Hydroxypropyl Trimonium Hydrolyzed | 0.1%-1% | Conditioner/ |
| Soy | | Quat |
| Propylene Glycol, Euterpe Oleracea, | 1% | Antiseptic, Anti- |
| (Acai) Fruit Extract, Lycium Barbarum | | inflammatory, |
| (Goji Berry) Fruit Extract, Punica | | Antioxidant, |
| Granatum (Pomegranate) Extract, | | Astringent |
| Diospyros, Kaki (Persimmon) Fruit | | |
| Extract | | |
| Benzylkonium Chloride | 0.2%-3% | Conditioner/ |
| | | Quat/Neut. |
| | | Agent |
| Sodium Hydroxide | 0.01% | Neutralizing |
| | | agent |
| Fragrance | 0.5% | Fragrance |

TABLE 7

| Ingredient | Percent | Functionality |
|---|---|---|
| Water | 76%-92% | Solvent, Mois- |
| | | turizer |
| Polyquaternium - 67 | 1%-2% | Conditioner/ |
| | | Quat |
| Phenoxyethanol, Methyloaraben, | 1% | Preservative |
| Butylparben, Ethylparaben, | | |
| Propylparaben, Isobutylpareben | | |
| Polyquaternium 22 | 0.1%-1% | Conditioner/ |
| | | Quat |
| Cetyl Trimethyl Ammonium Chloride | 1%-4% | Conditioner/ |
| | | Quat |
| Stearyl Alcohol | 0.1%-1.5% | Thickener |
| Cetyl Alcohol | 1%-3% | Thickener |
| Isocetyl Behanate | 2%-5% | Emollient |
| Glyceryl Stearate, PEG-100 Stearate | 1%-3.5% | Emulsifier |
| Hydroxypropyl Trimonium Hydrolyzed | 0.1%-0.85% | Conditioner/ |
| Soy | | Quat |
| Benzylkonium Chloride | 0.5%-2% | Conditioner/ |
| | | Quat/Neut. |
| | | Agent |
| Sodium Hydroxide | 0.01% | Neutralizing |
| | | agent |

TABLE 8

| Ingredient | Percent | Functionality |
|---|---|---|
| Water | 76%-91% | Solvent, Mois- |
| | | turizer |
| Polyquaternium - 67 | 1%-2% | Conditioner/ |
| | | Quat |
| Phenoxyethanol, Methyloaraben, | 1% | Preservative |
| Butylparben, Ethylparaben, | | |
| Propylparaben, Isobutylpareben | | |
| Polyquaternium 22 | 0.1%-1% | Conditioner/ |
| | | Quat |
| Cetyl Trimethyl Ammonium Chloride | 1%-3% | Conditioner/ |
| | | Quat |
| Stearyl Alcohol | 0.1%-1% | Thickener |
| Cetyl Alcohol | 1%-3% | Thickener |
| Isocetyl Behanate | 2%-5% | Emollient |
| Glyceryl Stearate, PEG-100 Stearate | 1%-4% | Emulsifier |
| Hydroxypropyl Trimonium Hydrolyzed | 0.1%-1% | Conditioner/ |
| Soy | | Quat |
| Propylene Glycol, Symphytum, | 1% | Skin healing, emollient, |
| Officinale Leaf Extract, Centaurea | | soothing, astringent, anti- |
| Cyanus Extract, Echinacea | | inflammatory, cleansing, |
| Angustifolia Leaf Extract, Silybum, | | antibacterial and |
| Marianum Extract | | antiseptic attributes |
| Benzylkonium Chloride | 0.5%-1.5% | Conditioner/ |
| | | Quat/Neut. |
| | | Agent |
| Sodium Hydroxide | 0.01% | Neutralizing |
| | | agent |
| Fragrance | 0.5% | Fragrance |

TABLE 9

| Ingredient | Percent | Functionality |
|---|---|---|
| Water | 77%-89% | Solvent, Mois- |
| | | turizer |
| Polyquaternium - 67 | 1%-2% | Conditioner/ |
| | | Quat |
| Phenoxyethanol, Methyloaraben, | 1% | Preservative |
| Butylparben, Ethylparaben, | | |
| Propylparaben, Isobutylpareben | | |
| Polyquaternium 22 | 0.1%-1% | Conditioner/ |
| | | Quat |
| Cetyl Trimethyl Ammonium Chloride | 1%-3% | Conditioner/ |
| | | Quat |
| Stearyl Alcohol | 0.1%-1% | Thickener |
| Cetyl Alcohol | 1%-3% | Thickener |
| Isocetyl Behanate | 2%-4% | Emollient |
| Glyceryl Stearate, PEG-100 Stearate | 2%-3% | Emulsifier |
| Hydroxypropyl Trimonium Hydrolyzed | 0.1%-1% | Conditioner/ |
| Soy | | Quat |
| Propylene Glycol, Symphytum, | 1% | Skin healing, emollient, |
| Officinale Leaf Extract, Centaurea | | soothing, astringent, anti- |
| Cyanus Extract, Echinacea | | inflammatory, cleansing, |
| Angustifolia Leaf Extract, Silybum, | | antibacterial and |
| Marianum Extract | | antiseptic attributes |
| Benzylkonium Chloride | 0.5%-2% | Conditioner/ |
| | | Quat/Neut. |
| | | Agent |
| Sodium Hydroxide | 0.01% | Neutralizing |
| | | agent |
| Fragrance | 0.5% | Fragrance |

TABLE 10

| Ingredient | Percent | Functionality |
|---|---|---|
| Water | 77%-89% | Solvent, Mois- |
| | | turizer |
| Polyquaternium - 67 | 1%-2% | Conditioner/ |
| | | Quat |
| Phenoxyethanol, Methylparaben, | 1% | Preservative |
| Butylparben, Ethylparaben, | | |
| Propylparaben, Isobutylpareben | | |
| Polyquaternium 22 | 0.1%-1% | Conditioner/ |
| | | Quat |
| Cetyl Trimethyl Ammonium Chloride | 1%-3% | Conditioner/ |
| | | Quat |
| Stearyl Alcohol | 0.1%-1% | Thickener |
| Cetyl Alcohol | 1%-3% | Thickener |
| Isocetyl Behanate | 2%-4% | Emollient |
| Glyceryl Stearate, PEG-100 Stearate | 2%-3% | Emulsifier |
| Hydroxypropyl Trimonium Hydrolyzed | 0.1%-1% | Conditioner/ |
| Soy | | Quat |
| Propylene Glycol, Symphytum, | 1% | Skin healing, emollient, |
| Officinale Leaf Extract, Centaurea | | soothing, astringent, anti- |
| Cyanus Extract, Echinacea | | inflammatory, cleansing, |
| Angustifolia Leaf Extract, Silybum, | | antibacterial and |
| Marianum Extract | | antiseptic attributes |
| Benzylkonium Chloride | 0.5%-2% | Conditioner/ |
| | | Quat/Neut. |
| | | Agent |
| Sodium Hydroxide | 0.01% | Neutralizing |
| | | agent |
| Fragrance | 0.5% | Fragrance |

I claim:

1. A skin softening and conditioning product formulation comprising one or more cationic agents in sufficient proportion to the remainder of the product such that, when applied to human skin, said cationic agents have a cationic strength sufficient to fully neutralize any negative electrostatic charge on the skin.

2. The skin softening and conditioning product formulation of claim 1 wherein one cationic agent is Celquat SC-240c (Polyquaternium-10).

3. The skin softening and conditioning product formulation of claim 2 wherein the cationic agent is in the range of 0 to 4 percent of the formulation by weight.

4. The skin softening and conditioning product formulation of claim 1 wherein one cationic agent is Merquat 100.

5. The skin softening and conditioning product formulation of claim 4 wherein the Merquat 100 is in the range of 0 to 11 percent of the formulation by weight.

6. The skin softening and conditioning product of claim 1 wherein one or more cationic agents are selected from the group consisting of:
Polyquaternium-22;
Polyquaternium-67;
Cetyl Trimethyl Ammonium Chloride;
Hydroxypropyl Trimonium Hydrolyzed Soy Protein; and
Benzalkonium Chloride.

7. The skin softening and conditioning product of claim 1 wherein said product is an aqueous solution.

8. The skin softening and conditioning product of claim 1 further comprising:
a) at least one preservative;
b) at least one thickener;
c) an emollient;
d) an emulsifier; and
e) a neutralizing agent to adjust pH as desired.

9. The skin softening and conditioning product of claim 8 wherein the at least one preservative is selected from the group consisting of:
Methyloaraben,
Butylparben,
Ethylparaben,
Propylparaben,
Isobutylpareben, and
Phenoxyethanol.

10. The skin softening and conditioning product of claim 8 wherein the at least one thickener is selected from the group consisting of Cetyl Alcohol, and Stearyl Alcohol.

11. The skin softening and conditioning product of claim 8 wherein the at least one emollient is selected from the group consisting of:
Isocetyl Behanate,
Water,
Hydrolyzed Algin,
Chlorella Vulgaris, and
C10-30 Cholesterol/Lanosterol Esters.

12. The skin softening and conditioning product of claim 8 wherein the emulsifier is Glyceryl Stearate and PEG-100 Stearate.

13. The skin softening and conditioning product of claim 8 wherein the neutralizing agent is sodium hydroxide.

14. The skin softening and conditioning product of claim 1 further comprising an antioxidant.

15. The skin softening and conditioning product of claim 1 further comprising an astringent.

16. The skin softening and conditioning product of claim 1 further comprising an agent to promote skin healing, having cleansing, antibacterial and antiseptic attributes.

17. The skin softening and conditioning product of claim 1 further comprising a fragrance.

18. The skin softening and conditioning product of claim 6 wherein the amount of Polyquaternium-22 ranges from 0.1% to 1.2% by weight.

19. The skin softening and conditioning product of claim 6 wherein the amount of Polyquaternium-67 ranges from 0.5% to 3% by weight.

20. The skin softening and conditioning product of claim 6 wherein the amount of Cetyl Trimethyl Ammonium Chloride ranges from 1% to 4% by weight.

21. The skin softening and conditioning product of claim 6 wherein the amount of Hydroxypropyl Trimonium Hydrolyzed Soy Protein ranges from 0.1% to 1% by weight.

22. The skin softening and conditioning product of claim 6 wherein the amount of Benzalkonium Chloride ranges from 0.2% to 3% by weight.
